(19) United States
(12) Patent Application Publication
(10) Pub. No.: US 2025/0166825 A1
(43) Pub. Date: May 22, 2025

(54) PATHOGENICITY DETERMINATION DEVICE, PATHOGENICITY DETERMINATION METHOD, MACHINE LEARNING METHOD, AND LEARNED MODEL GENERATION METHOD

(71) Applicant: Hitachi, Ltd., Tokyo (JP)

(72) Inventors: Sachiho ABE, Tokyo (JP); Tomohiro YASUDA, Tokyo (JP)

(21) Appl. No.: 18/929,245

(22) Filed: Oct. 28, 2024

(30) Foreign Application Priority Data
Nov. 22, 2023 (JP) 2023-198198

(51) Int. Cl. G16H 50/20

(52) U.S. Cl. CPC G16H 50/20

(57) ABSTRACT

A pathogenicity determination device of the present disclosure includes: an input device that receives inputs of genetic mutation information indicating a genetic mutation, and genetic mutation-related information related to the genetic mutation information; a processor that estimates a first score related to presence or absence of a pathological significance of the genetic mutation and a second score related to strength or sufficiency of evidence related to the genetic mutation, based on the genetic mutation information and the genetic mutation-related information; and an output device that outputs the estimated first score and the estimated second score.

### CROSS REFERENCE TO RELATED APPLICATIONS

The present application claims priority from Japanese patent application JP 2023-198198 filed on Nov. 22, 2023, the entire content of which is hereby incorporated by reference into this application.

### BACKGROUND OF THE INVENTION

### 1. Field of the Invention

The present disclosure relates to a pathogenicity determination device, a pathogenicity determination method, a machine learning method, and a learned model generation method.

### 2. Description of the Related Art

In cancer genomic medicine, an expert panel is carried out after a genomic test result is returned. In the expert panel, the pathological significance (Oncogenic, Benign, or Variants for Unknown Significance (VUS)) of genetic mutations of patients is discussed by experts such as doctors. The pathological significance is comprehensively determined based on a variety of evidence, and its determination requires time, labor, and specialized knowledge. Further, there is no clear criterion for the determination of the pathological significance, thus causing a difference in the determination results among experts. Accordingly, the determination of the pathological significance is dependent on individual skills, and a great burden is imposed on specific experts.

Meanwhile, various systems and artificial intelligence (AI) systems for determining the pathological significance of genetic mutations have been recently developed. For example, US 2017/0316149 A recites a technique for classifying DNA variants into five categories including “Pathogenic”, “Highly Pathogenic”, “Variants for Unknown Significance (VUS)”, “Highly Benign”, and “Benign Variant” based on a rule-based scoring system.

### SUMMARY OF THE INVENTION

Even in a case where the pathological significance of genetic mutations is determined by AI, it is assumed that the pathological significance is finally confirmed by an expert. Since it is difficult to understand the degree of accuracy of the result determined only by the determination of the pathological significance using AI, a significant burden is imposed on the expert in confirming the determination result. There is a number of reasons for determining the pathological significance by AI. Particularly, there are different variations of the reason to be determined as VUS. Further, there are a mutation in which the determination of the pathological significance in multiple evidence conflicts and it is difficult to determine the pathological significance (the accuracy of the determination result is low) and a mutation in which the determination of the pathological significance in multiple evidence conflicts and it is easy to determine the pathological significance (the accuracy of the determination result is high).

In the technique of US 2017/0316149 A, the estimation result of genetic mutation (VUS) of which presence or absence of the pathological significance is unknown includes a mix of VUS of which genetic significance is unknown simply due to lack of evidence and VUS of which presence or absence of the pathological significance is unable to be determined although there is evidence. Therefore, in order to extract the VUS having evidence, it is necessary to manually confirm and determine the genetic information and the information related to the corresponding genetic mutation.

Hence, the present disclosure provides a technique for reducing a personnel burden for confirming the validity of the estimation result of the pathological significance.

In order to solve the above problems, a pathogenicity determination device of the present disclosure includes: an input device that receives inputs of genetic mutation information indicating a genetic mutation of a patient, and genetic mutation-related information related to the genetic mutation information; a processor that estimates a first score related to the presence or absence of pathological significance of the genetic mutation and a second score related to the strength or sufficiency of evidence related to the genetic mutation, based on the genetic mutation information and the genetic mutation-related information; and an output device that outputs the estimated first score and the estimated second score.

Additional characteristics related to the present disclosure will be apparent from the description of the present specification and the attached drawings. Aspects of the present disclosure are achieved and realized by elements, combinations of various elements, and aspects of the following detailed description, and the appended scope of claims. The description herein is merely exemplary and does not limit the scope of claims or application examples of the present disclosure in any sense.

According to the technique of the present disclosure, it is possible to reduce a personnel burden for confirming the validity of the estimation result of the pathological significance. Problems, configurations, and effects other than those described above will be clarified by the description of embodiments below.

### BRIEF DESCRIPTION OF THE DRAWINGS

FIG. 1 is a configuration diagram of a pathogenicity determination device according to a first embodiment;

FIG. 2A is a schematic diagram illustrating an example of learning data;

FIG. 2B is a schematic diagram illustrating an example of correct answer data;

FIG. 2C is a schematic diagram illustrating an example of test data;

FIG. 3 is a schematic diagram illustrating an example of a mutation score estimated for test data;

FIG. 4 is a schematic diagram of a setting screen according to the first embodiment;

FIG. 5 is a schematic diagram of an output screen according to the first embodiment;

FIG. 6A is a flowchart of a learning method of a learning model according to the first embodiment;

FIG. 6B is a flowchart of a pathogenicity determination method using the learned learning model according to the first embodiment;

FIG. 7 is a schematic diagram of a setting screen according to a second embodiment;

FIG. 8 is a schematic diagram of an output screen according to the second embodiment;

FIG. 9 is a flowchart of a pathogenicity determination method using a learned learning model according to the second embodiment;

FIG. 10 is a schematic diagram of a setting screen according to a third embodiment;

FIG. 11 is a schematic diagram of an output screen according to the third embodiment;

FIG. 12 is a flowchart of a pathogenicity determination method using a learned learning model according to the third embodiment;

FIG. 13 is a configuration diagram of a pathogenicity determination device according to a fourth embodiment;

FIG. 14 is a schematic diagram of a setting screen according to the fourth embodiment; and

FIG. 15 is a flowchart of a learning method of a learning model according to the fourth embodiment.

### DESCRIPTION OF THE PREFERRED EMBODIMENTS

### First Embodiment

### Pathogenicity Determination Device

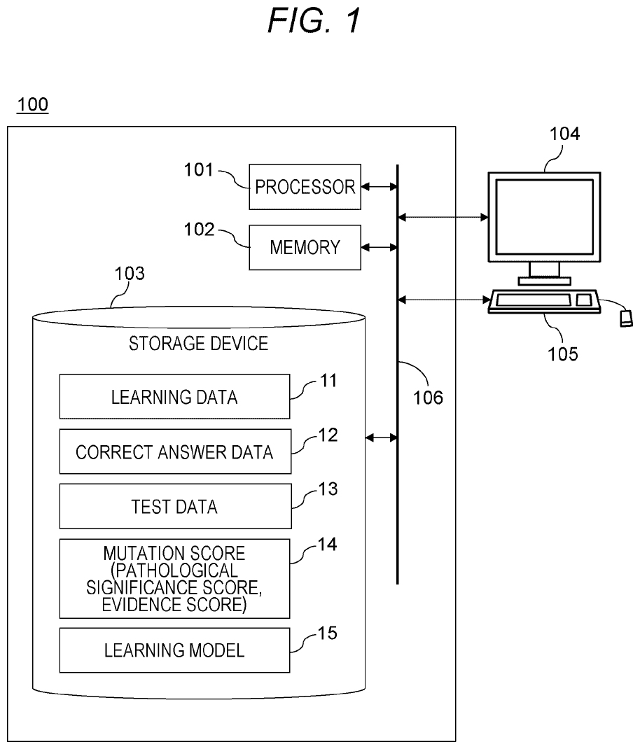

FIG. 1 is a configuration diagram of a pathogenicity determination device 100 according to a first embodiment. The pathogenicity determination device 100 is a device for determining the pathological significance (Oncogenic, Benign, or VUS) of a genetic mutation in a genomic test result of a patient. The pathogenicity determination device 100 includes, for example, a personal computer, a server device, a smartphone, a tablet, an office computer, and a general-purpose machine (main frame). As illustrated in FIG. 1, the pathogenicity determination device 100 includes a processor 101, a memory 102, a storage device 103, a display device 104, an input device 105, and a bus 106 that connects the processor 101, the memory 102, the storage device 103, the display device 104, and the input device 105.

The processor 101 realizes the function of the pathogenicity determination device 100 by executing a program developed in the memory 102. As the processor 101, for example, a CPU or a GPU can be used. The number of processors 101 is not limited to one, and the function of the pathogenicity determination device 100 may be implemented by a plurality of processors. The memory 102 includes a ROM and a RAM.

The storage device 103 stores learning data 11, correct answer data 12, test data 13, a mutation score 14, and a learning model 15. The learning data 11 is data for the learning model 15 to learn through machine learning or data already learned. The correct answer data 12 is data that is the correct answer to the output of the learning model 15, and is associated with the learning data 11 by a common ID or the like. The test data 13 is data as an estimation target of the pathological significance using the learning model 15, and includes information on the genetic mutation in the genomic test result of the patient. Details of the learning data 11 and the test data 13 will be described later.

The mutation score 14 is a pathological significance score and an evidence score of each genetic mutation of the patient in the test data 13, estimated (output) by the learning model 15. The pathological significance score is a score indicating an estimation result: Oncogenic or Benign and its accuracy. The evidence score is a score indicating the strength or sufficiency of evidence. As described later, the pathological significance score and the evidence score are expressed as numerical values, and expressed as (y₁, y₂)=(pathological significance score, evidence score). Note that the correct answer data 12 is obtained by converting the pathological significance as a correct answer determined by an expert for the learning data 11 into a mutation score.

The learning model 15 is a machine learning model for estimating the pathological significance of each genetic mutation in the genomic test result of the patient. The learning model 15 is a supervised learning model trained by the learning data 11 associated with the correct answer data 12. The learning model 15 is constructed such that the pathological significance score and the evidence score are each independently estimated and output using the test data 13 as an input. Alternatively, the learning model for estimating the pathological significance score and the learning model for estimating the evidence score may be separately provided. As a machine learning algorithm of the learning model 15, for example, an arbitrary algorithm such as XGBoost or a neural network can be adopted.

Although not illustrated, basic information of the patient is also stored in the storage device 103. The basic information of the patient includes, for example, patient identification number (ID), age, gender, and cancer type. The basic information of the patient is associated with the test data 13 and the mutation score 14 by, for example, a common ID.

The display device 104 is, for example, a liquid crystal display. The input device 105 is, for example, a mouse or a keyboard. A touch panel may be used as both the display device 104 and the input device 105.

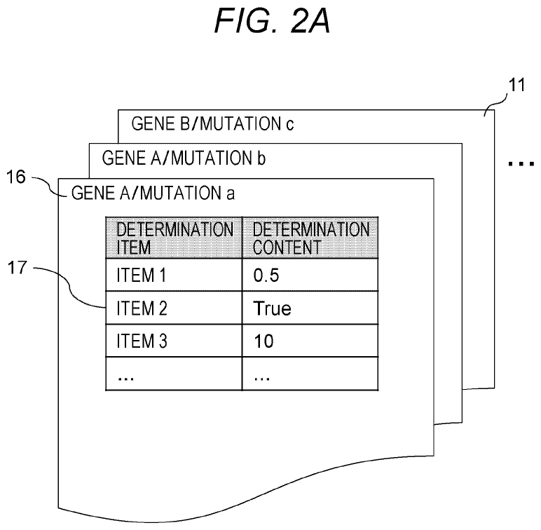

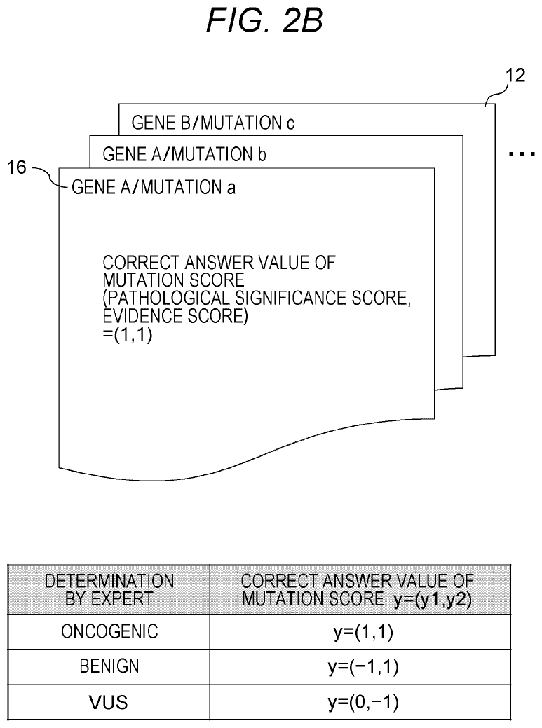

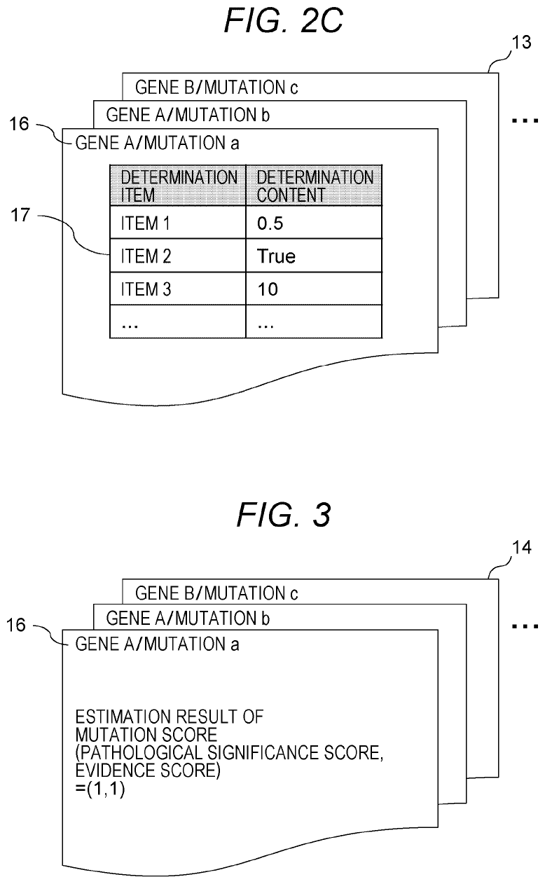

FIG. 2A is a schematic diagram illustrating an example of the learning data 11. As illustrated in FIG. 2A, the learning data 11 includes genetic mutation information 16 and genetic mutation-related information 17. The genetic mutation information 16 indicates a genetic mutation. In the present embodiment, the genetic mutation information 16 is represented as “gene A/mutation a”, “gene A/mutation b”, “gene B/mutation c”, or the like. The genetic mutation-related information 17 is associated with each piece of the genetic mutation information 16.

The genetic mutation-related information 17 is represented in the form of a table including a plurality of determination items for determining the pathological significance and determination contents of the determination items. The determination items include, for example, known information regarding the genetic mutation information 16 that can be acquired from an external public known mutation information database (such as ClinVar or COSMIC). Specific examples of the determination items include, for example, the polymorphic allele frequency, the determination result of the pathological significance in the mutation information database, presence or absence of possibility of canceration when a genetic mutation is present, amino acid information, the number of reports for each cancer type, and the position of the domain in which the mutation is present. The determination items may be a combination of clinical information (e.g. age, cancer type) in each case and information in the public known mutation information database. For example, the cancer type in the clinical information can be combined with information such as the number of reports for each cancer type.

The determination content can be expressed in an arbitrary data format. The determination content is represented by, for example, a numerical value corresponding to the determination item, True or False, a label, and a determination result of pathological significance in the public known mutation information database. For example, when the determination item is “polymorphic allele frequency”, the determination content may be a numerical value such as “0.5”. When the determination item is “whether the pathological significance of the mutation has been reported”, the determination content may be “True” or “False”. In a case where the determination item is “determination of pathological significance in the public known mutation information database”, the determination content may be “Oncogenic”, “Benign”, “VUS”, “Likely Oncogenic”, “Likely Benign”, or the like. The determination items of the genetic mutation-related information 17 and their determination contents are used in estimation of the mutation score. For the determination items and their determination contents, it is determined which pathological significance is supported. In comprehensive consideration of the determination result, and it is determined which pathological significance the genetic mutation has.

FIG. 2B is a schematic diagram illustrating an example of the correct answer data 12. As illustrated in FIG. 2B, the correct answer data 12 includes correct answer values of mutation scores (a pathological significance and an evidence score). The correct answer data 12 is created for each piece of the genetic mutation information 16.

A lower part of FIG. 2B shows a method of creating the correct answer data 12. Correct answer values: mutation scores y=(y₁, y₂) in the correct answer data 12 are determined based on the result of the pathological significance of the genetic mutation information 16 determined by the expert. When the expert determines the pathological significance of the genetic mutation information 16 as “Oncogenic”, the pathological significance score and the evidence score are 1 and 1, respectively. When the expert determines the pathological significance of the genetic mutation information 16 as “Benign”, the pathological significance score and the evidence score are −1 and 1, respectively. When the expert determines the pathological significance of the genetic mutation information 16 as “VUS”, the pathological significance score and the evidence score are 0 and −1, respectively.

FIG. 2C is a schematic diagram illustrating an example of the test data 13. As illustrated in FIG. 2C, the format and content of the test data 13 are similar to the format and content of the learning data 11. The genetic mutation information 16 included in the test data 13 may be only the genetic mutation detected in the genomic test of the patient. As described above, the learning data 11 and the test data 13 include all information used to calculate the mutation score 14.

FIG. 3 is a schematic diagram illustrating an example of the mutation score 14 estimated for the test data 13. The mutation score 14 (pathological significance score and evidence score) is estimated by the learning model 15 for each piece of the genetic mutation information 16. The pathological significance score and the evidence score are each indicated by a numerical value in a range of −1 to 1. The processor 101 determines the pathological significance of the genetic mutation information 16 from the values of the pathological significance score and the evidence score.

An example of the pathogenicity determination method by the pathological significance score will be described. When the pathological significance score is a positive value, the result is determined as “Oncogenic”. When the pathological significance score is a negative value, the result is determined as “Benign”. When the pathological significance score is 0, the result is determined as “VUS”. As an absolute value of the pathological significance score is larger, the accuracy of the determination result of the pathological significance is higher. Thus, a threshold for determining the pathological significance by the pathological significance score is defined as 0, the presence or absence of the pathological significance and the VUS can be determined. Alternatively, the threshold of the pathological significance score can be any value. Specifically, for example, thresholds j and k (j>k) of two pathological significance scores can be provided. At this time, in the case of j<pathological significance score, the result is determined as “Oncogenic”. In the case of k<pathological significance score <j, the result is determined as VUS. In the case of pathological significance score <k, the result is determined as “Benign”. For example, the thresholds j and k of the pathological significance scores may be set to j=0.5 and k=−0.5, respectively. A user can set the thresholds of the pathological significance scores on a setting screen to be described later.

The evidence score indicates the strength and sufficiency of evidence that are the basis of the determination result of the pathological significance. In a case where the evidence score is a positive value, the evidence score becomes higher as the number of evidence as the basis of the determination result of the pathological significance is larger, or the number of evidence largely contributed to the determination result of the pathological significance is larger. The term “largely contributed to the determination result of the pathological significance” refers to, for example, evidence that can clearly indicate whether a genetic mutation is “Oncogenic” or “Benign”. When the evidence score is a negative value, the result is determined as “VUS”. Thus, the pathological significance of the genetic mutation information 16 can be determined based on the estimated mutation score 14.

The correct answer data of the pathological significance score and the evidence score and the threshold for determining the pathological significance are not limited to those described above, and other mathematical formulas may be used, or any modification is possible.

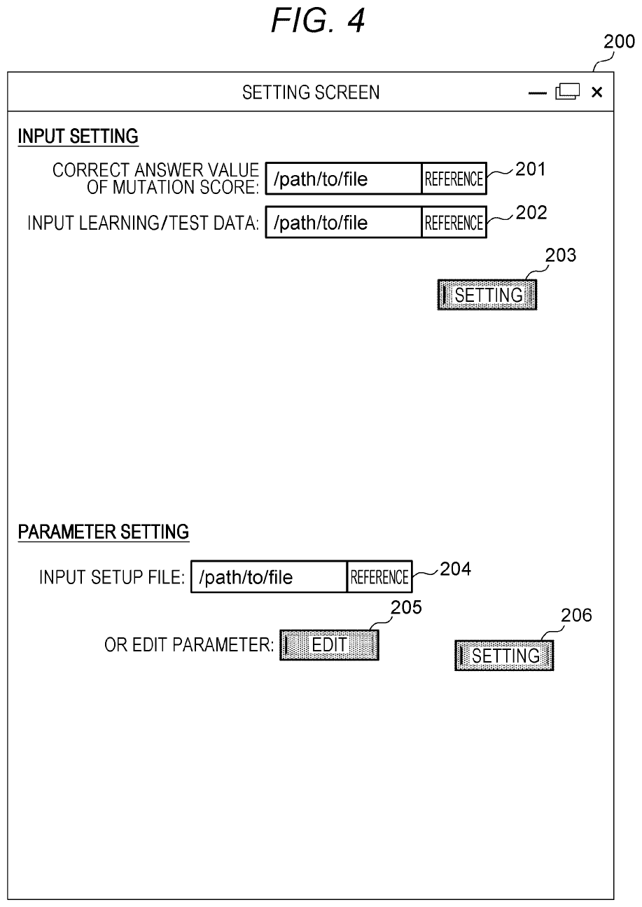

FIG. 4 is a schematic diagram of a setting screen 200 according to the first embodiment. The setting screen 200 is used for setting input data for causing the learning model 15 to learn, setting input data to be an estimation target of the pathological significance of genetic mutation using the learned learning model 15, and setting internal parameters of the learning model 15.

As illustrated in FIG. 4, the setting screen 200 includes a box 201, a box 202, a setting button 203, a box 204, an edit button 205, and a setting button 206. In the box 201, the user can designate a file of the correct answer data 12 used for learning the learning model 15. In the box 202, the user can designate a file of the learning data 11 used for learning the learning model 15 or a file of the test data 13 as an estimation target of the learning model 15. A designated file can be input to the processor 101 (the memory 102) by the setting button 203. When the user clicks the box 201 and the box 202, a desired file can be selected from the files stored in the storage device 103, and a path of the file can be designated accordingly.

In the box 204, the user can designate a file for setting the internal parameters of the learning model 15. The edit button 205 allows the user to edit the parameters. In the setting button 206, a setting of the designated file or the edited parameters can be input. The parameters are, for example, hyperparameters. The parameters may be parameters optimized by prior learning.

In a case where a determination item important in the pathogenicity determination is known in the parameter editing via the edit button 205, the user may weight the internal parameters of the learning model 15 depending on the magnitude of contribution to the determination of the genetic mutation-related information (determination item).

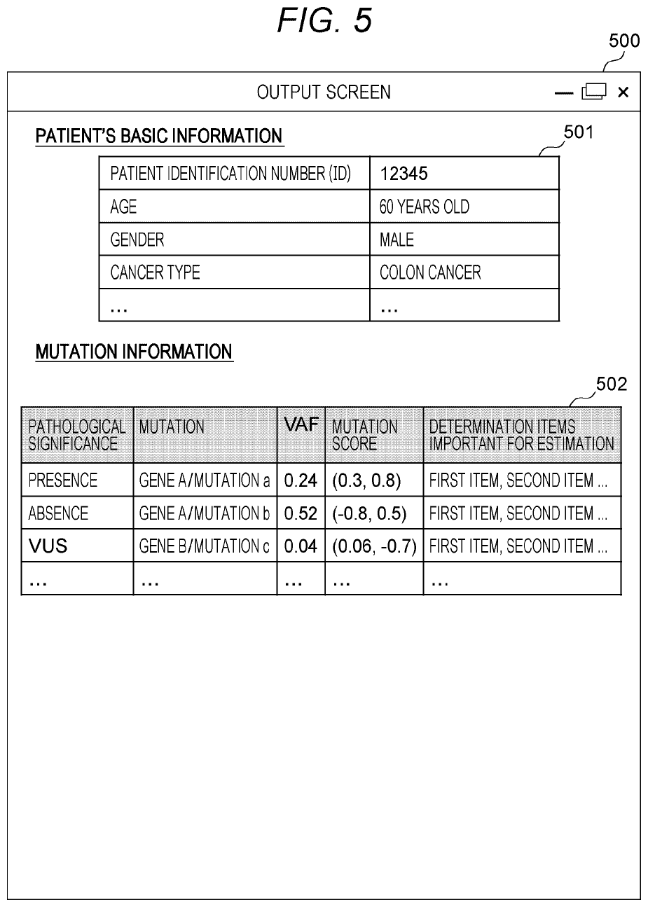

FIG. 5 is a schematic diagram of an output screen 500 according to the first embodiment. The output screen 500 is displayed on the display device 104. The output screen 500 includes patient's basic information 501 and mutation information 502. The patient's basic information 501 and the mutation information 502 are represented in a table format. The patient's basic information 501 includes patient identification number (ID), age, gender, cancer type, and the like.

The mutation information 502 includes a result of pathological significance (Oncogenic, Benign, or VUS) determined by the processor 101 using the learning model 15, genetic mutation information, variant allele frequency ((VAF):ratio of cells whose genetic mutation is detected), an estimated mutation score, and determination items important for estimation. In the column of determination items important for estimation, the determination items strongly contributed to the estimation are ranked. Such ranking of the determination items can be determined by a predetermined algorithm included in the learning model 15 such as XGBoost. Based on the mutation information 502, the user can confirm the mutation score estimated for each piece of the genetic mutation information and the determination result of the pathological significance.

### Learning Stage

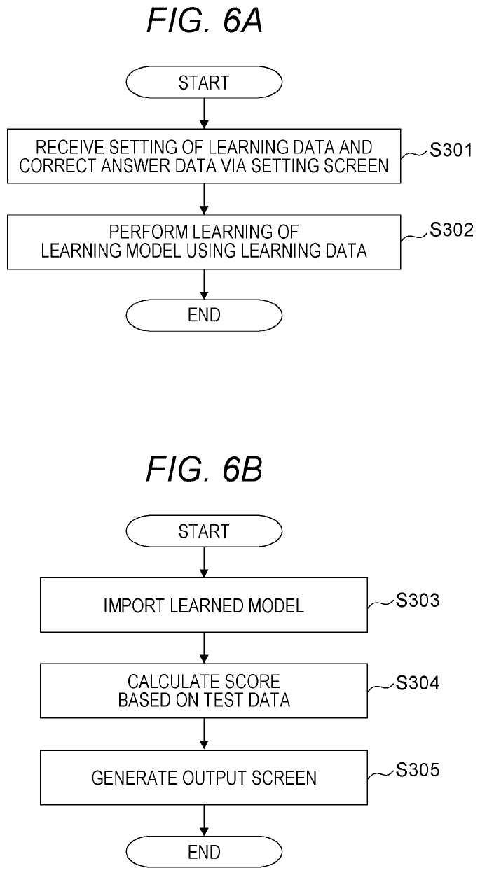

FIG. 6A is a flowchart of a learning method of the learning model 15 according to the first embodiment. The learning method is executed by the processor 101 and includes the following steps S301 and S302. In step S301, the processor 101 receives inputs of the learning data 11, the correct answer data 12 of the mutation score, and the setting of the internal parameters from the user via the setting screen 200. In step S302, the processor 101 learns the learning model 15 using the learning data 11 to which the correct answer data 12 is assigned as a correct answer label and the set internal parameters. The processor 101 stores the learned learning model 15 in the storage device 103.

### Test Stage

FIG. 6B is a flowchart of a pathogenicity determination method using the learned learning model 15 according to the first embodiment. The pathogenicity determination method is executed by the processor 101 and includes the following steps S303 to S305.

In step S303, the processor 101 imports the learned learning model 15 from the storage device 103 into the memory 102.

In step S304, the processor 101 receives an input of the test data 13 from the user via the setting screen 200. Thereafter, the processor 101 inputs the test data 13 to the learning model 15, and acquires the mutation score 14 (pathological significance score and evidence score) which is the output (estimation result) of the learning model 15. Further, the processor 101 determines the pathological significance of the genetic mutation information 16 based on the mutation score 14. The processor 101 stores the mutation score 14 and the determination result of the pathological significance in the storage device 103.

In step S305, the processor 101 generates the output screen 500 including the mutation score 14, the determination result of the pathological significance, and the patient's basic information, and causes the display device 104 to display the output screen 500.

Note that the processor 101 may further learn the learning model 15 using the mutation score 14 acquired in step S304. The learning of the learning model 15 using such an estimation result can also be performed each time the mutation score 14 is estimated.

### Estimation Method Using Mathematical Formula

The method of estimating the mutation score 14 by machine learning has been described above. Alternatively, the mutation score 14 can be calculated by an evidence-based mathematical formula. In this case, the processor 101 determines whether each determination item and its determination content supports the determination as Oncogenic, Benign, or VUS, and uses the determination item and its determination content for calculation of the pathological significance score and the evidence score.

The pathological significance score y₁ is represented by the following Formula (1).

$$\begin{matrix}[ Math . 1 ] & \\ \hat{{y}_{1}} = σ \{ {\sum }_{{x : evidence of P}^{}} ( a ( x ) - {w}_{1} \cdot n ( x ) ) + {b}_{1} ) - {\sum }_{x : e v i dence of B} ( a ( x ) - {w}_{1} \cdot n ( x ) ) + {b}_{1} ) \} & ( 1 )\end{matrix}$$

In Formula (1), σ represents a sigmoid function. x represents an evidence level. P represents a determination item supporting the determination as “Oncogenic” and its determination content. B represents a determination item supporting the determination as “Benign” and its determination content. a(x) represents a constant of the evidence level, and takes a numerical value of 5 (x is high) to 1 (x is low). a(x) is defined in accordance with rule-based guidelines for each evidence. w₁ represents a weight. n represents the number of pieces. b₁ represents a hyperparameter of a baseline.

As in Formula (1), the pathological significance score y₁ is calculated as a value obtained by normalizing a difference between the sum of the evidence levels of the determination items supporting the determination as “Oncogenic (P)” and the sum of the evidence levels of the determination items supporting the determination as “Benign (B)” by a sigmoid function (σ).

The evidence score y₂ is represented by the following Formula (2).

$$\begin{matrix}[ Math . 2 ] & \\ \hat{{y}_{2}} = σ \{ {\sum }_{x : e v i d ence of P} ( a ( x ) - {w}_{1} \cdot n ( x ) ) + {b}_{1} ) + {\sum }_{x : e v i d ence of B} ( a ( x ) - {w}_{1} \cdot n ( x ) ) + {b}_{1} ) - {\sum }_{x : e v i d ence of VUS} ( a ( x ) - {w}_{2} \cdot n ( x ) ) + {b}_{2} ) \} & ( 2 )\end{matrix}$$

In Formula (2), σ represents a sigmoid function. x represents an evidence level. P represents a determination item supporting the determination as “Oncogenic” and its determination content. B represents a determination item supporting the determination as “Benign” and its determination content. VUS represents a determination item supporting the determination as “VUS” and its determination content. a(x) represents a constant of the evidence level, and takes a numerical value of 5 (x is high) to 1 (x is low). a(x) is defined in accordance with rule-based guidelines for each evidence. w₁ and w₂ each represent a weight. n represents the number of pieces. b₁ and b₂ each represent a hyperparameter of a baseline.

As in Formula (2), the evidence score y₂ is calculated as a value obtained by subtracting the sum of the evidence levels of the determination items supporting the determination as “VUS”, from the total of the sum of the evidence levels of the determination items supporting the determination as “Oncogenic (P)” and the sum of the evidence levels of the determination items supporting the determination as “Benign (B)”, and normalizing the resultant value by the sigmoid function (σ).

The mathematical formula for calculating the pathological significance score and the mathematical formula for calculating the evidence score are not limited to the mathematical formulas described above, and other mathematical formulas may be used, or any modification is possible.

Information described in a known literature regarding determination of the pathological significance of a genetic mutation can also be used for calculating the mutation score 14. Examples of the information described in the known literature include information in a polymorphism database such as gnomAD, evidence of an effect on canceration in vitro or in vivo, pathogenic evidence of mutation, the number of reported cases of the same amino mutation and the same position mutation in a database such as Cancer Hotspots or COSMIC, and a determination result of pathological significance by a calculation tool. Examples of the known literatures include P. Horak et al., Genetics in Medicine (2022) 24, 986-998.

When the pathological significance is determined using the mathematical formulas as described above, ranking of “determination items important for estimation” in the mutation information 502 on the output screen 500 can be performed based on the level of the evidence level.

### Summary of First Embodiment

As described above, the pathogenicity determination device 100 according to the first embodiment includes: the input device 105 that receives inputs of the genetic mutation information 16 indicating a genetic mutation and the genetic mutation-related information 17 related to the genetic mutation information 16; the processor 101 that estimates a pathological significance score (a first score) related to the presence or absence of the pathological significance of the genetic mutation and an evidence score (a second score) related to the strength or sufficiency of evidence related to the genetic mutation based on the genetic mutation information 16 and the genetic mutation-related information 17; and the display device 104 (output device) that outputs the estimated pathological significance score and the estimated evidence score (the first score and the second score).

According to the pathogenicity determination device 100, the accuracy of the determination result of the pathological significance is secured by the pathological significance score (the first score) related to the presence or absence of the pathological significance. Further, the interpretation of a genetic mutation for which the presence or absence of the pathological significance is unable to be determined is indicated by the evidence score (the second score) indicating the sufficiency of evidence. Furthermore, outputting and visualizing the pathological significance score and the evidence score enables the user (expert) to easily identify the genetic mutation to be preferentially confirmed and discussed. This process allows for reduction of the operation for the expert to confirm all the genetic information.

In a case where there is a therapeutic drug for the genetic mutation, the expert can determine whether to recommend a therapeutic method using the drug. The accuracy of the determination as “Oncogenic” as indicated by the pathological significance score can support that determination.

### Second Embodiment

In the first embodiment described above, it has been described that the mutation score 14 estimated by the learning model 15 is displayed as a numerical value on the output screen 500. Additionally or alternatively, the estimated mutation score 14 may be plotted on a two-dimensional plane as described in the second embodiment. The configuration of the pathogenicity determination device according to the second embodiment is the same as the configuration of the first embodiment, and thus the description of the configuration will not be repeated.

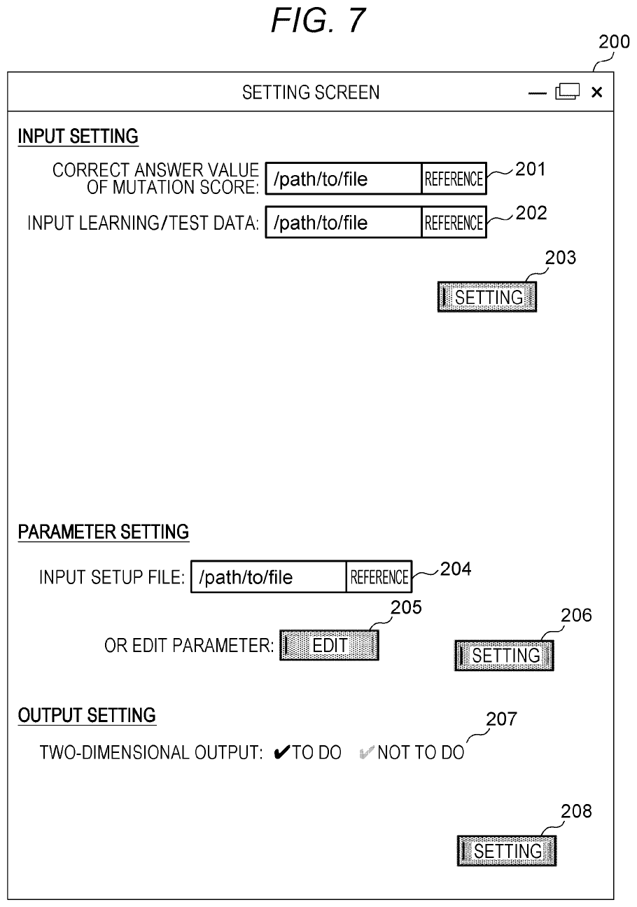

FIG. 7 is a schematic diagram of the setting screen 200 according to the second embodiment. The setting screen of the present embodiment includes a selection button 207 and a setting button 208, in addition to the content of the setting screen of the first embodiment. The selection button 207 allows the user to select whether or not to output a mutation score on a two-dimensional plane on the output screen 500. The selection button 207 can check either “to do” or “not to do” the two-dimensional output. The setting as to whether or not to output the two-dimensional plane selected by the selection button 207 can be input to the processor 101 (the memory 102) via the setting button 208.

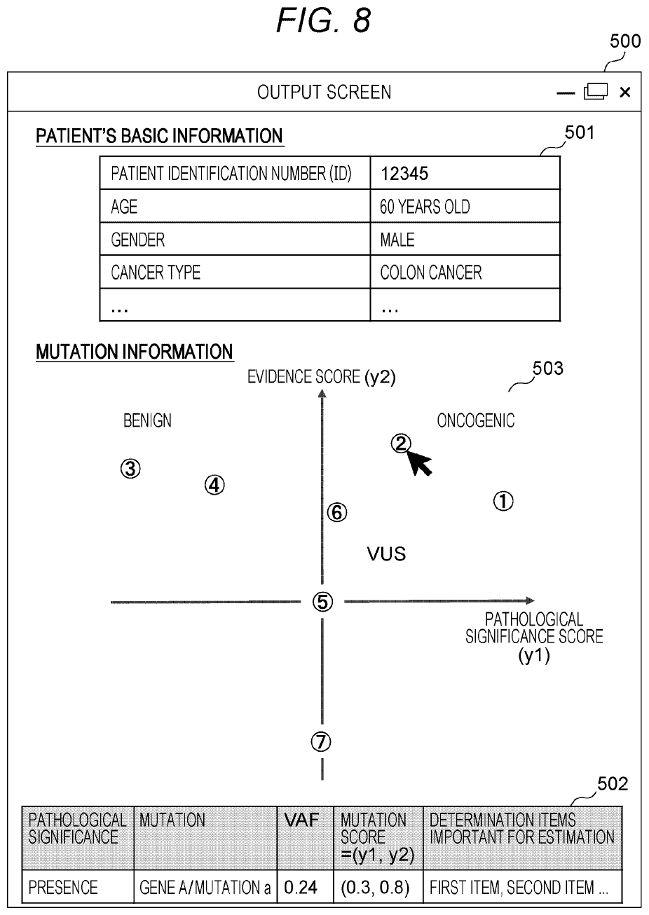

FIG. 8 is a schematic diagram of the output screen 500 according to the second embodiment. The output screen 500 of the present embodiment further includes a graph 503 of the mutation score (two-dimensional plane), in addition to the content of the output screen of the first embodiment. The graph 503 is a graph obtained by plotting the mutation scores corresponding to each piece of the genetic mutation information, which have been calculated in step S304 as described above. The horizontal axis represents the pathological significance score (y₁), and the vertical axis represents the evidence score (y₂).

In the graph 503, mutation scores (No. 1 to No. 7) for 7 pieces of genetic mutation information are plotted. When the user clicks (selects) an arbitrary plot, information on the genetic mutation of the plot (mutation score, pathogenicity determination items important for estimation, and VAF) is displayed in the table of the mutation information 502. In FIG. 8, the second plot is clicked. In a case where none of the plots is clicked (selected), all the detected genetic mutation information and the estimation result of the pathological significance based on the genetic mutation information are displayed as in the mutation information 502 illustrated in FIG. 5.

In the graph 503, mutation scores of genetic mutations estimated in the past can also be plotted. In this case, the user confirms the mutation information 502 regarding the mutation scores estimated in the past, and thus the user can use the mutation scores close to the plotted mutation scores as a reference to determine the pathological significance.

### Test Stage

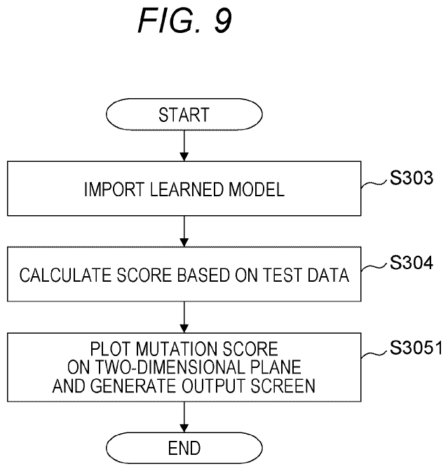

FIG. 9 is a flowchart of a pathogenicity determination method using the learned learning model 15 according to the second embodiment. The pathogenicity determination method according to the second embodiment is different from the pathogenicity determination method according to the first embodiment in that step S3051 is executed instead of step S305. In step S3051, the processor 101 generates the output screen 500 including the graph obtained by plotting the mutation scores calculated in step S304 on the two-dimensional plane. Then, the processor 101 causes the display device 104 to display the output screen 500. Note that the learning method of the learning model 15 is the same as the learning method of the learning model 15 according to the first embodiment.

### Summary of Second Embodiment

As described above, in the pathogenicity determination device according to the second embodiment, the display device 104 outputs a two-dimensional graph in which the pathological significance score and the evidence score (the first score and the second score) are plotted. As a result, the user can visually and easily confirm the determination result of the pathological significance of the genetic mutation.

### Third Embodiment

In the second embodiment described above, it has been described that the graph with the estimated mutation scores plotted on the two-dimensional plane is output. As described in the third embodiment below, each region indicating the determination as Oncogenic, Benign, or VUS may be further illustrated on the graph.

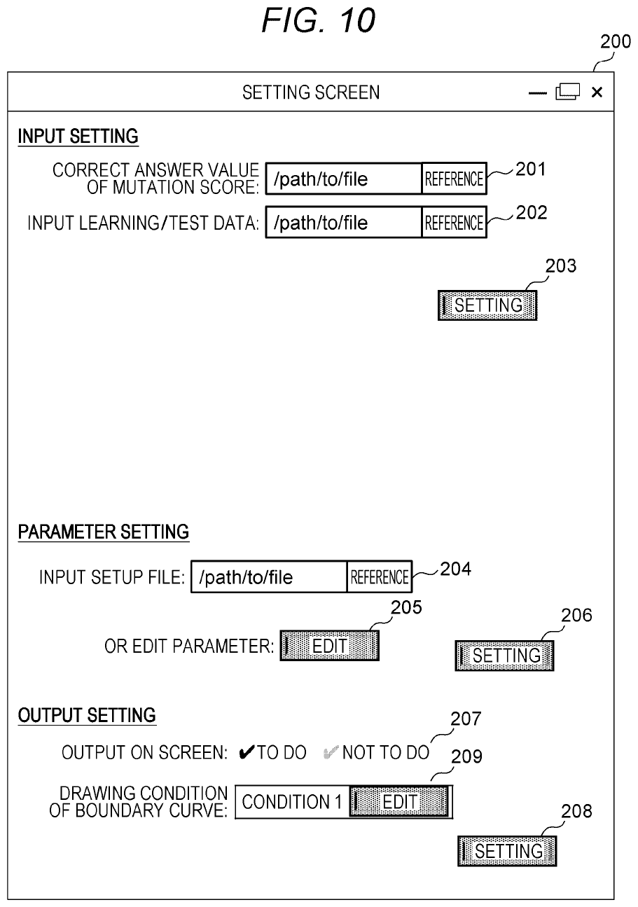

FIG. 10 is a schematic diagram of the setting screen 200 according to the third embodiment. The setting screen of the present embodiment further includes a drawing condition button 209 for editing a drawing condition of a boundary curve, in addition to the content of the setting screen of the second embodiment. For example, the drawing condition of the boundary curve can be configured such that the form of the function of the boundary of the determination as “Oncogenic”, “Benign”, or “VUS” can be input. Further, the setting screen 200 of the present embodiment can be configured such that a drawing condition of a “region to be preferentially discussed” can be input.

The drawing condition of the boundary curve may be set to draw a region where the ratio correctly predicted in the learning data 11 is 100%. At this time, the ratio correctly predicted in the learning data 11 is represented by the following Formula (3).

$$\begin{matrix}[ Math . 3 ] & \\ Correctly predicted ratio = \frac{Correct answer data determined as Oncogenic and estimation result determined as Oncogenic}{Estimation result determined as Oncogenic} & ( 3 )\end{matrix}$$

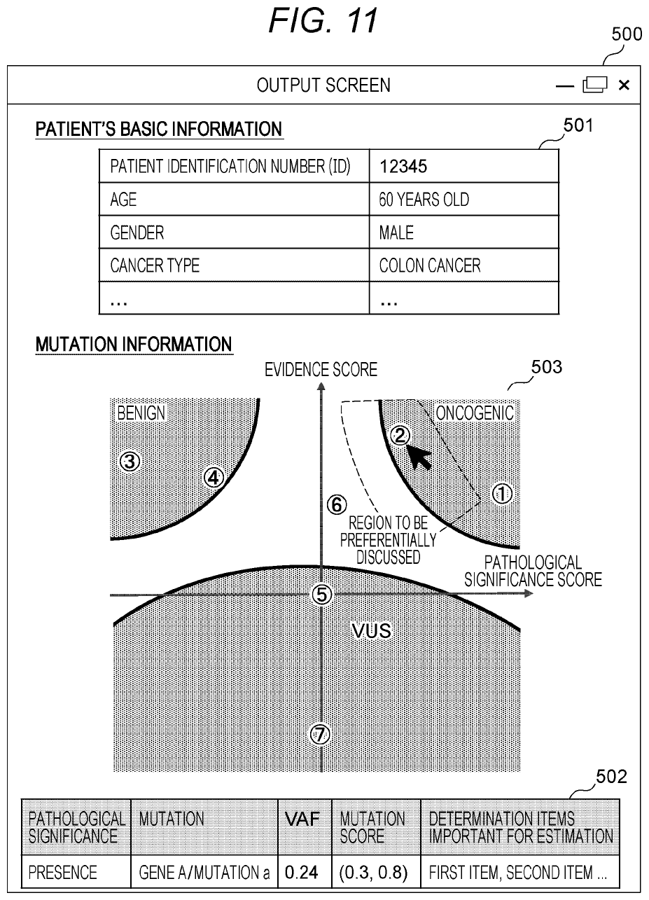

FIG. 11 is a schematic diagram of the output screen 500 according to the third embodiment. As illustrated in FIG. 11, boundary curves of regions are superimposed on the graph 503 of the mutation scores illustrated in FIG. 8. The regions separated by the boundary curves can be indicated by different colors. Further, a portion near a boundary of each of the regions is emphasized as the “region to be preferentially discussed”. The mutation to be preferentially confirmed and discussed is a mutation in which both the determination item supporting the determination as Oncogenic and the determination item supporting the determination as Benign are present, and there is no positive reason to be determined as VUS. Further, in a case where a plot is included in the “region to be preferentially discussed”, it may be displayed that human discussion is needed via the output device. As described above, experts can preferentially discuss the mutation which has both the evidence as “Oncogenic” and the evidence as “Benign” near the boundary line and for which the experts are hesitating to make judgment. Meanwhile, for a plot with high accuracy of estimation of the pathological significance, the result estimated by the pathogenicity determination device 100 can be directly adopted. Thus, this allows for reduction of the time and effort for discussion and the personnel burden.

Boundary curves are illustrated along with, for example, a region where 80% of the included data is correct and a region where 60% of the included data is correct, in addition to a region where 100% of the included data is correct, and may be illustrated to form contour lines. In other words, it is possible to draw boundary lines of a plurality of regions indicating a plurality of predetermined ratios among the ratios correctly predicted in the learning data 11.

### Test Method

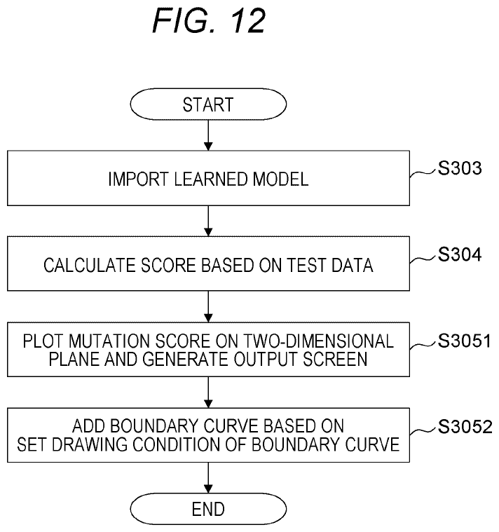

FIG. 12 is a flowchart of a pathogenicity determination method using the learned learning model 15 according to the third embodiment. The pathogenicity determination method according to the third embodiment is different from the pathogenicity determination method according to the second embodiment in that step S3052 is further included. In step S3052, the processor 101 generates a boundary curve based on the drawing condition of the boundary curve set by the drawing condition button 209, and generates a diagram superimposed on the graph generated in step S3051. Note that the learning method of the learning model 15 is the same as the learning method of the learning model 15 according to the first embodiment.

### Summary of Third Embodiment

As described above, in the pathogenicity determination device according to the third embodiment, a boundary line of a region indicating a ratio at which the pathological significance is correctly determined in the learning data 11 is 100% (predetermined ratio) is drawn on a two-dimensional graph obtained by plotting the pathological significance score and the evidence score. Then, a plot near the boundary line (genetic mutation having a predetermined relationship with the boundary line) is displayed as the “region to be preferentially discussed”, thereby prompting the user to confirm the determination of the pathological significance.

### Fourth Embodiment

In the first embodiment described above, it has been described that the user selects the files of the learning data 11 and the test data 13 and uses the files as the inputs of the learning model 15. Alternatively, as described in the fourth embodiment, the pathogenicity determination device may create the learning data 11 and the test data 13.

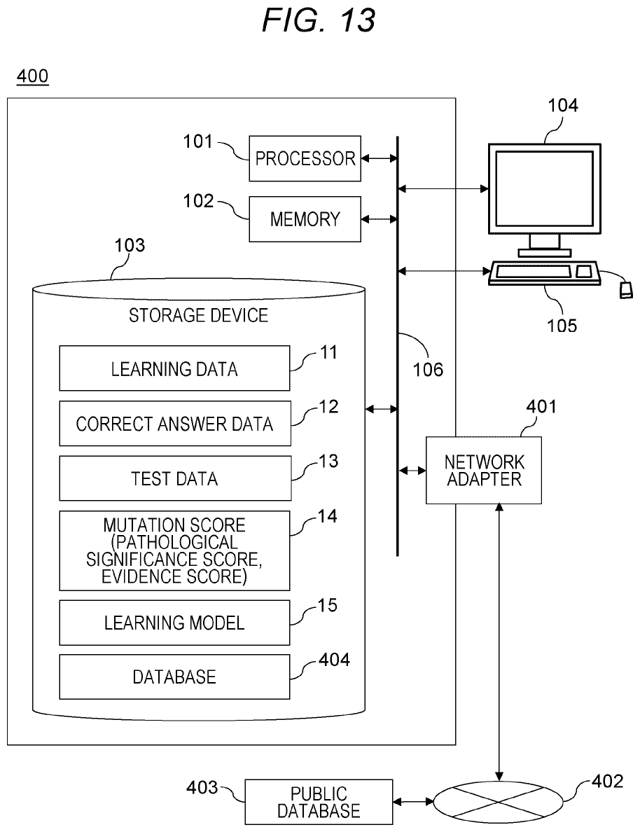

FIG. 13 is a configuration diagram of a pathogenicity determination device 400 according to the fourth embodiment. The pathogenicity determination device 400 further includes a network adapter 401, and is configured to be accessible to an external public database 403 via a network 402. The public database 403 is a public known mutation information database such as ClinVar or COSMIC. In the fourth embodiment, the processor 101 is configured to create the learning data 11 and the test data 13 using the information acquired by accessing the public database 403 and store the learning data and the test data in the storage device 103. A method of creating the learning data 11 and the test data 13 will be described later.

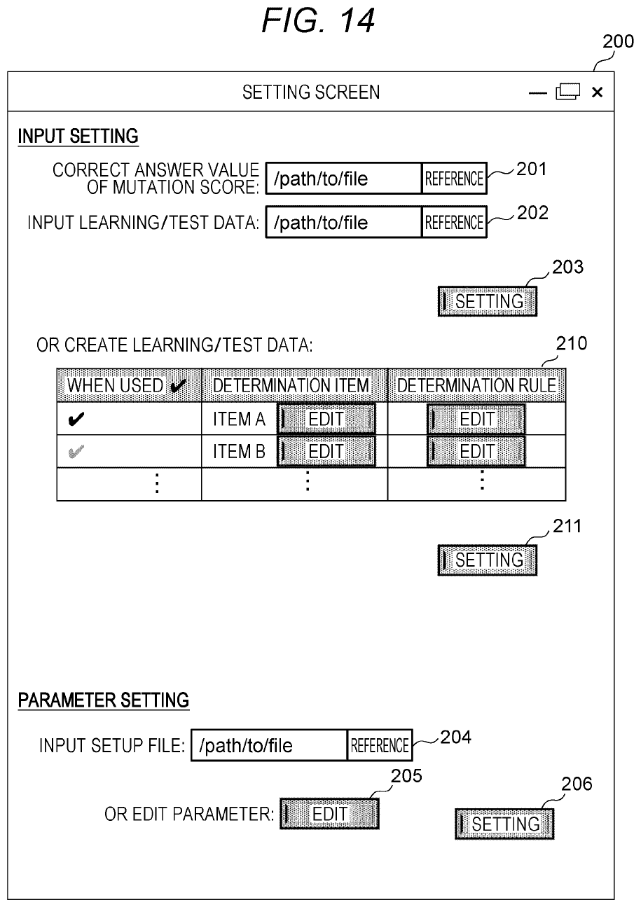

FIG. 14 is a schematic diagram of the setting screen 200 according to the fourth embodiment. The setting screen of the present embodiment includes a table 210 for creating the learning data 11 and the test data 13 and a setting button 211, in addition to the content of the setting screen of the first embodiment. The table 210 includes a check field for selecting whether to use the determination item, an edit column of the determination item, and an edit field of the determination rule. Note that the determination item and the determination rule may be set by default, or can be optionally edited by the user.

The determination item set in the table 210 correspond to the determination item in the genetic mutation-related information 17 described with reference to FIGS. 2A and 2C. Examples of the determination item include the polymorphic allele frequency and the number of reported cases in the public database 403.

The determination rule is described as a rule for extracting, from the public database 403, information as the determination content in the genetic mutation-related information 17 described with reference to FIGS. 2A and 2C. As an example of the determination rule, a rule such as “The public database is searched and the allele frequency of mutation with matching mutation name is input” can be set for the polymorphic allele frequency. A rule such as “In the mutation having the same mutation name, another item: the number of cases registered in the cancer type of the case is extracted and input” can be set for the number of reported cases.

The user inputs necessary information in the table 210, and then the user clicks the setting button 211. When the setting button 211 is clicked, the processor 101 executes a predetermined program to create the learning data 11 and the test data 13 based on the information input to the table 210. The learning data 11 and the test data 13 are created in the format described with reference to FIGS. 2A and 2C. Depending on which of the setting button 203 and the setting button 211 is clicked, the processor 101 can determine whether to use a file designated via the box 202 as the learning data 11 and the test data 13 or to create the learning data 11 and the test data 13 using data input by the user via the table 210.

### Learning Method

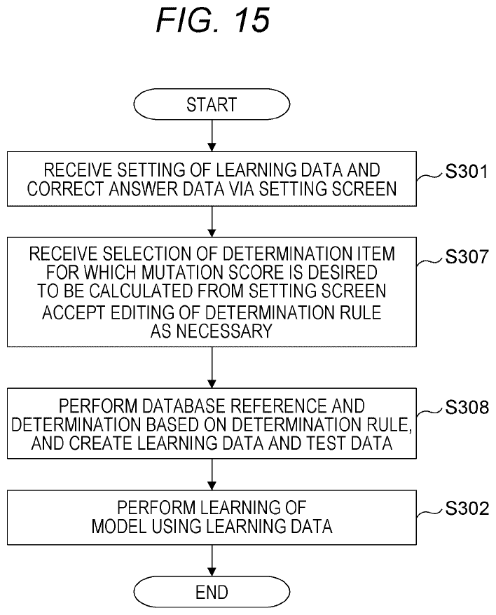

FIG. 15 is a flowchart of a learning method of the learning model 15 according to the fourth embodiment. The learning method according to the fourth embodiment is different from the learning method according to the first embodiment in that steps S307 and S308 are further included between steps S301 and S302.

In step S307, the processor 101 receives an input (check) of a determination item for which a mutation score is desired to be calculated, from the user via the setting screen 200. Editing of the determination rule from the user via the setting screen 200 is accepted as necessary. In step S308, the processor 101 refers to and determines the public database 403 based on the determination rule, and creates learning data and test data. The pathogenicity determination method using the learned learning model 15 is the same as the pathogenicity determination method according to the first embodiment.

### Modified Example of Fourth Embodiment

In the fourth embodiment, it has been described that the processor 101 accesses the external public database 403 via the network 402 to acquire information necessary for creating the learning data 11 and the test data 13. Alternatively, a vendor of the pathogenicity determination device 400 may create a database similar to the public database 403 in advance, and store the database as the database 404 (see FIG. 13) in the storage device 103. In this case, in step S308 described above, the processor 101 is configured to refer to the database 404. In this case, the network adapter 401 is unnecessary.

Alternatively, the processor 101 may download information available in the public database 403 and store the information as the database 404 in the storage device 103.

### Summary of Fourth Embodiment

As described above, in the pathogenicity determination device 400 according to the fourth embodiment, the processor 101 creates, as the genetic mutation-related information 17, data including a determination item for determining the pathological significance and a determination content of the determination item, based on information available from the external public database 403 (public known mutation information database). Thus, the pathogenicity determination device 400 is responsible for collecting information necessary for determining the pathological significance of the genetic mutation, thereby reducing labor for the vendor or the user to prepare the learning data 11 and the test data 13.

### Modified Example

The present disclosure is not limited to the above-described embodiments, and includes various modified examples. For example, the above-described embodiments have been described in detail in order to describe the present disclosure in an easily understandable manner, and all the described configurations are not necessarily included. Furthermore, part of one embodiment can be replaced with a configuration of another embodiment. Alternatively, the configuration of another embodiment can be added to the configuration of one embodiment. Alternatively, as for part of the configuration of each of the embodiments, part of the configuration of another embodiment can be added, deleted, or displaced.

What is claimed is:

1. A pathogenicity determination device comprising:
an input device that receives inputs of genetic mutation information indicating a genetic mutation, and genetic mutation-related information related to the genetic mutation information;
a processor that estimates a first score related to presence or absence of a pathological significance of the genetic mutation and a second score related to strength or sufficiency of evidence related to the genetic mutation, based on the genetic mutation information and the genetic mutation-related information; and
an output device that outputs the estimated first score and the estimated second score.

2. The pathogenicity determination device according to claim 1, wherein the processor further determines the pathologic significance of the genetic mutation based on a combination of the first score and the second score.

3. The pathogenicity determination device according to claim 1, wherein the output device outputs a two-dimensional graph in which the first score and the second score are plotted.

4. The pathogenicity determination device according to claim 1, wherein the processor further creates, as the genetic mutation-related information, data including a determination item for determining the pathological significance and a determination content of the determination item, based on information available from a public known mutation information database outside the pathogenicity determination device.

5. The pathogenicity determination device according to claim 1, further comprising a storage device that stores a learning model constructed to estimate the first score and the second score using the genetic mutation information and the genetic mutation-related information as inputs,
wherein the processor inputs the genetic mutation information and the genetic mutation-related information to the learning model, and outputs the first score and the second score.

6. The pathogenicity determination device according to claim 5, wherein the processor further causes the learning model to learn using learning data in which the genetic mutation information and the genetic mutation-related information are associated with the first score and the second score as correct answer values.

7. The pathogenicity determination device according to claim 6, wherein the output device draws a boundary line of a region indicating a predetermined ratio at which the pathological significance is correctly determined in the learning data, on a two-dimensional graph in which the first score and the second score are plotted.

8. The pathogenicity determination device according to claim 7, wherein the output device highlights a region including the genetic mutation having a predetermined relationship with the boundary line.

9. The pathogenicity determination device according to claim 7, wherein the output device further draws a boundary line of a plurality of regions indicating a plurality of the predetermined ratios on the two-dimensional graph.

10. The pathogenicity determination device according to claim 1, wherein the genetic mutation-related information includes presence or absence of possibility of canceration when the genetic mutation is present, a number of reported cases, or amino acid information.

11. A pathogenicity determination method executed by a processor of a pathogenicity determination device, comprising:
receiving, from an input device of the pathogenicity determination device, inputs of genetic mutation information indicating a genetic mutation, and genetic mutation-related information related to the genetic mutation information;
estimating a first score related to presence or absence of a pathological significance of the genetic mutation and a second score related to strength or sufficiency of evidence related to the genetic mutation, based on the genetic mutation information and the genetic mutation-related information; and
outputting the estimated first score and the estimated second score to an output device.

12. A machine learning method of a learning model applied to a pathogenicity determination device that determines a pathological significance of a genetic mutation,
the machine learning method comprising:
causing a processor to acquire genetic mutation information indicating the genetic mutation and genetic mutation-related information related to the genetic mutation information;
causing the processor to acquire correct answer data of a first score related to presence or absence of a pathological significance of the genetic mutation and a second score related to strength or sufficiency of evidence related to the genetic mutation; and
causing the processor to learn the learning model using the genetic mutation information, the genetic mutation-related information, and the correct answer data as learning data.

13. A learned model generation method to be applied to a pathogenicity determination device that determines a pathological significance of a genetic mutation,
the learned model generation method comprising:
causing a processor to acquire genetic mutation information indicating the genetic mutation and genetic mutation-related information related to the genetic mutation information;
causing the processor to acquire correct answer data of a first score related to presence or absence of a pathological significance of the genetic mutation and a second score related to strength or sufficiency of evidence related to the genetic mutation; and
causing the processor to construct the learned model such that the first score and the second score are estimated using the genetic mutation information, the genetic mutation-related information, and the correct answer data as learning data, and using the genetic mutation information and the genetic mutation-related information as inputs.
